(19) United States
(12) Patent Application Publication
(10) Pub. No.: US 2019/0059993 A1
(43) Pub. Date: Feb. 28, 2019

(54) VIBRATING CATHETER FOR RADIO-FREQUENCY (RF) ABLATION

(71) Applicant: Biosense Webster (Israel) Ltd., Yokneam (IL)

(72) Inventors: Christopher Thomas Beeckler, Brea, CA (US); Andres Claudio Altmann, Haifa (IL)

(73) Assignee: Biosense Webster (Israel) Ltd., Yokneam (IL)

(21) Appl. No.: 15/692,173

(22) Filed: Aug. 31, 2017

(51) Int. Cl. A61B 18/14

(52) U.S. Cl. CPC A61B 18/1492; A61B 2018/00577

(57) ABSTRACT

A medical instrument includes a shaft, multiple electrodes and a vibration generator. The shaft is configured for insertion into a body of a patient. The multiple electrodes are fitted at a distal end of the shaft and are configured to deliver Radio Frequency (RF) energy for ablation at multiple respective locations in tissue. The vibration generator is configured to vibrate the multiple electrodes for providing cooling to the tissue in a vicinity of the locations.

### FIELD OF THE INVENTION

The present invention relates generally to medical probes, and particularly to multi-electrode RF ablation catheters.

### BACKGROUND OF THE INVENTION

Various known invasive medical instrument designs apply ablative RF energy to a patient's tissue, coupled with means to minimize side effects of the ablation. For example, U.S. Patent Application Publication 2002/0147446 describes an electrosurgical apparatus including an electrode adapted to deliver RF energy to a tissue. A manipulator in operable connection with the electrode, vibrates the electrode so as to at least diminish adherence of tissue to the electrode. A controller in communication with the RF power source and the manipulator is adapted to control the operation of the manipulator and the electrode.

As another example, U.S. Patent Application Publication 2008/0161795 describes an ablation catheter which controls the temperature and reduces the coagulation of biological fluids on an electrode of a catheter. The device also prevents the impedance rise of tissue in contact with the electrode and maximizes the potential energy transfer to the tissue, thereby allowing an increase in the lesion size produced by the ablation. The electrode includes passages positioned to allow saline flow out of an inner cavity of the electrode. This fluid flow is pulsatile to increase turbulence, reducing areas of stagnant flow, and produces a desired cooling effect.

U.S. Patent Application Publication 2009/0287209 describes an ablation catheter which has an electrode that can electrically cauterize a living body tissue at the tip side of a catheter. The electrode is characterized in that the vibration and/or rotation of the electrode is controllable according to the temperature of the cauterization portion.

U.S. Pat. No. 5,100,423 describes an ablation catheter comprising a plurality of helically-shaped cutting wires attached to the shaft to form a cutting basket. As the catheter is moved through a vessel, such as a blood vessel, it separates obstructing matter on the inner surface of a vessel cavity from the inner surface. The proximal ends of the cutting wires extend from the shaft through a vibratory transducer. Electrical current is applied to heat the wires to facilitate separating obstructing matter from a vessel surface. Furthermore, the vibration transducer vibrates the cutting wires to further aid in separating plaque and obstruction from soft tissue such as the intima layer of the wall of a blood vessel.

In “Radiofrequency ablation with a vibrating catheter: A new method for electrode cooling,” Medical Engineering and Physics, May 2016, volume 38, issue 5, pages 458-467, Yu et al. describe vibrations of a catheter that are hypothesized to disturb blood flow around the electrode, leading to increased convective cooling of the electrode. The results show that under conditions of no flow, electrode temperatures decreased with increasing vibration frequency. In the presence of vibrations, electrode temperatures decreased under conditions of low flow velocity, but not under those of high flow velocity. A disturbed flow around the vibrating catheter was demonstrated, and flow velocity around the catheter increased with higher-frequency vibrations.

### SUMMARY OF THE INVENTION

An embodiment of the present invention provides a medical instrument including a shaft, multiple electrodes and a vibration generator. The shaft is configured for insertion into a body of a patient. The multiple electrodes are fitted at a distal end of the shaft and are configured to deliver Radio Frequency (RF) energy for ablation at multiple respective locations in tissue. The vibration generator is configured to vibrate the multiple electrodes for providing cooling to the tissue in a vicinity of the locations.

In some embodiments, the medical instrument includes one or more temperature sensors fitted at the distal end of the shaft and configured to measure one or more respective temperatures. The medical instrument also includes a processor configured to read the measured temperatures from the temperature sensors and to command the vibration generator to vibrate the multiple electrodes responsively to one or more of the read temperatures.

In some embodiments, the processor is configured to adjust at least one of an amplitude and a frequency with which the vibration generator vibrates the multiple electrodes, responsively to one or more of the read temperatures. In an embodiment, the processor is configured to activate or deactivate the vibration generator responsively to one or more of the read temperatures.

In another embodiment, the multiple electrodes are configured to deliver the RF energy in a sequence of pulses, and the medical instrument includes a processor configured to command the vibration generator to vibrate the multiple electrodes in synchronization with the pulses.

In another embodiment, the medical instrument includes a cooling irrigation device fitted at the distal end of the shaft, wherein the cooling irrigation device is configured to deliver saline solution. In some embodiments, the medical instrument includes a processor configured to command the vibration generator to vibrate the multiple electrodes in coordination with delivery of the saline solution from the irrigation device.

In an embodiment, the medical instrument includes a basket ablation device, a multi-arm ablation device, or a balloon ablation device, which is fitted at the distal end of the shaft and includes the multiple electrodes.

In another embodiment, the vibration generator includes an electroactive polymer. In some embodiments, the vibration generator includes an oscillating solenoid. In an embodiment, the oscillating solenoid is configured to be driven by an external alternating magnetic field.

In another embodiment, the vibration generator is fitted at the distal end of the shaft. In an embodiment, the vibration generator is fitted at a proximal end of the shaft. In an embodiment, the vibration generator is configured to vibrate the multiple electrodes by vibrating a sheath in which the shaft is inserted.

There is additionally provided, in accordance with an embodiment of the present invention, a method including inserting a shaft of a medical instrument into a body of a patient. Radio Frequency (RF) energy is delivered from multiple electrodes fitted at a distal end of the shaft, for ablating multiple respective locations in a tissue. The multiple electrodes are vibrated for providing cooling to tissue in a vicinity of the locations.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based ablation system, in accordance with an embodiment of the present invention;

FIG. 2 is a schematic, pictorial illustration of a catheter distal end comprising RF electrodes and a vibration generator, in accordance with an embodiment of the present invention;

FIG. 3 is a schematic graph illustrating a sequence of synchronized RF energy pulses and vibration pulses, in accordance with an embodiment of the present invention; and

FIG. 4 is a flow chart that schematically illustrates a method for controlling tissue temperature during ablation, in accordance with an embodiment of the present invention.

### DETAILED DESCRIPTION OF EMBODIMENTS

### Overview

Embodiments of the present invention that are described herein provide improved methods and instruments for providing cooling to tissue subjected to RF ablation energy.

In some embodiments, a multi-electrode ablation device is fitted at the distal end of a shaft of a medical instrument, such as a catheter, to perform a cardiac RF ablation process. A vibration generator is also fitted in the catheter. During the ablation procedure, the generator vibrates the distal end of the shaft and/or at least part of the ablation device itself to improve the local movement of blood. A processor jointly controls the RF ablation pulses and the vibration.

The purpose of improving the local movement of blood is to dissipate undesired heat that is otherwise hard to evacuate, which the ablation electrodes may generate. Movement of blood may mean in this description flow of blood, turbulence, any way of mixing of hotter and colder blood, or any other type of blood motion caused by the vibration that contributes to reducing tissue temperature.

Some of the embodiments of the disclosed invention may provide solutions to shortcomings of other cooling solutions, for example irrigation. Irrigation techniques suffer from several limitations. Technically, it is complicated to implement comprehensive schemes of irrigation with complex high-power multi-electrode ablation geometries. Clinically, there are scenarios in which irrigation of saline is not permitted, or have limited access to tissue. Adding or switching to vibration-induced cooling may thus turn advantageous in various occasions.

In some embodiments, the controlling processor applies a closed-loop control of the vibration of the catheter distal end to provide additional cooling to the ablated tissue and its surroundings. In some embodiments, the processor receives temperature readings from one or more temperature sensors fitted at the distal end, and commands the vibration generator to adjust its vibration-amplitude and/or vibration-frequency responsively to the temperature readings, so as to control the temperature. In another embodiment, the processor activates or deactivates the vibration generator responsively to the temperature readings as to control the temperature.

In an embodiment, the ablation device is a “basket ablation device” fitted at the distal end of a catheter. The basket ablation device comprises a plurality of RF ablation electrodes, a plurality of temperature sensors, and a vibration generator. One or more of the temperature reading serve as an input parameter to the processor for the closed-loop control of the operation of the vibration. Such scheme provides additional flexibility to the basket ablation device cooling apparatus, as explained below. The basket ablation device may additionally comprise one or more irrigation apertures for delivering saline solution that cools the tissue during the ablation.

In some embodiments, the vibration of the distal end of the shaft is coupled with duty cycling the RF power. Such synchronization between the RF pulses and the vibration may allow the endocardial surface of the lesion to cool but still remain hot at depth. Each pulse or vibration would slightly increase the temperature at depth while the surface would oscillate between blood temperature and a temperature insufficient to coagulate blood.

One motivation of the disclosed technique is to enable the cooling of tissue otherwise in firm contact with the electrodes throughout the ablation and thus less accessible to other cooling techniques. Any slight displacement of the electrode due to vibration may allow blood to come into contact briefly with the hot tissue which allows for evacuation of heat from its surface. Thus, proper timing of vibration with the application of RF power may allow efficient cooling of the tissue at its instantaneous peak surface temperature.

The disclosed technique has thus potential clinical advantages, for example, over existing solutions in preventing blood coagulation at zones where irrigation has no access to, or regions of low blood flows where irrigation may not be permitted. Moreover, the vibration may be synergistically combined with irrigation, allowing a mixture of cold saline and cold blood access to otherwise inaccessible tissue hot spots.

For the reasons described above, the disclosed system might be especially beneficial when applying the RF ablative energy simultaneously through a plurality of electrodes. Such mode of operation requires both tight control of the electrical power and of tissue temperatures, making efficient irrigation difficult to realize. The disclosed RF ablation instruments may thus, for example, enable implementing simplified irrigation systems to support such mode of operation. Additionally, more reliable suppression of tissue over-heating can potentially reduce other clinical side-effects, such as collateral thermal damage to other nearby unrelated soft tissues.

### System Description

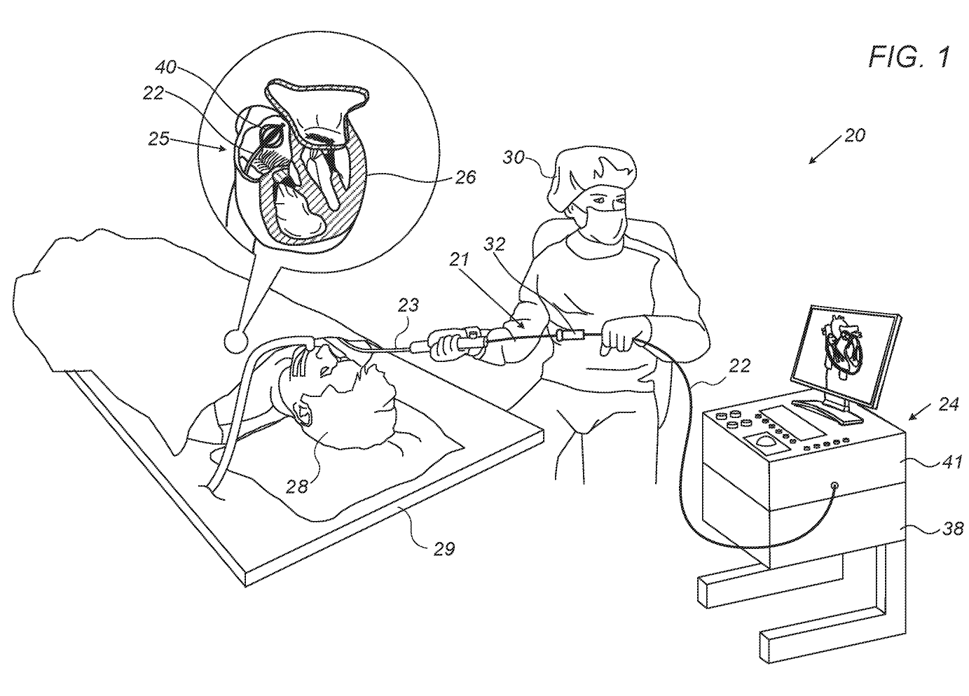

FIG. 1 is a schematic, pictorial illustration of a catheter-based ablation system 20, in accordance with an embodiment of the present invention. System 20 comprises a catheter 21, wherein a shaft 22 of the catheter is inserted into a heart 26 of a patient 28 through a sheath 23. The proximal end of catheter 21 is connected to a control console 24. In the embodiment described herein, catheter may be used for any suitable therapeutic and/or diagnostic purposes, such as electrical sensing and/or ablation of tissue in heart 26.

Console 24 comprises a processor 41, typically a general-purpose computer, with suitable front end. Console 24 comprises also a control unit 38 for receiving signals from catheter 21, as well as for applying RF energy via catheter 21 to ablate tissue in heart 26 and for controlling the other components of system 20. Processor 41 may be configured to jointly control the application of ablation pulses and vibration.

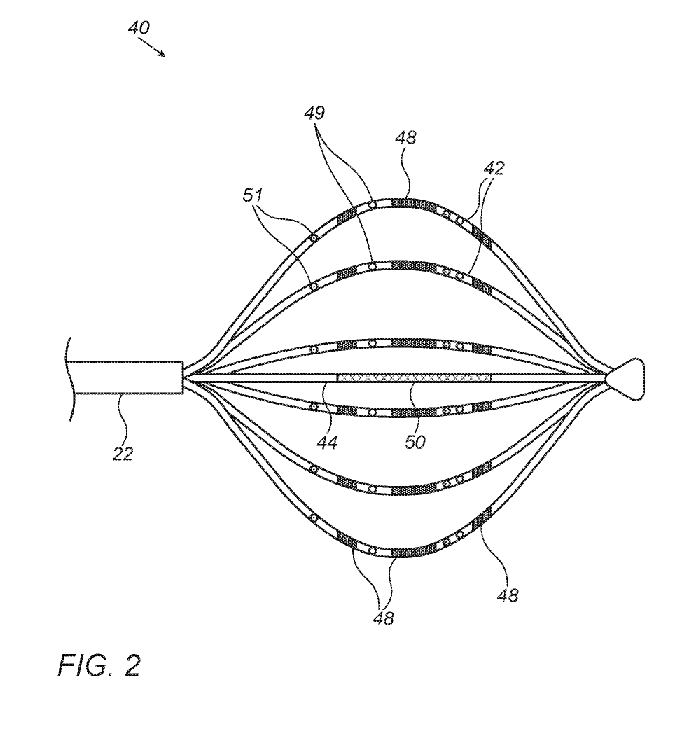

A physician 30 inserts shaft 22 through the vascular system of patient 28 lying on a table 29. Catheter 21 comprises a basket ablation device 40 fitted at the distal end of shaft 22. During the insertion of shaft 22, basket ablation device 40 is maintained in a collapsed configuration by sheath 23. By containing device 40 in a collapsed configuration, sheath 23 also serves to minimize vascular trauma along the way to target location. Physician 30 navigates basket ablation device 40 to a target location in heart 26 by manipulating shaft 22 using a manipulator 32 near the proximal end of the catheter and/or deflection from the sheath 23. Once the distal end of shaft 22 has reached the target location, physician 30 retracts sheath 23, letting basket ablation device 40 to expand. The physician then operates console 24 so as sense signals and apply ablation energy through ablation electrodes 48 (seen in FIG. 2) to the tissue at the target location.

Although the pictured embodiment relates specifically to the use of a basket ablation device for ablation of heart tissue, the elements of system 20 and the methods described herein may alternatively be applied in controlling ablation using other sorts of multi-electrode ablation devices, such as lasso, balloon, and multi-arm ablation devices.

### Enhanced Cooling During RF Ablation Using Vibration

FIG. 2 is a schematic, pictorial illustration of basket ablation device 40 comprising a vibration generator 50, in accordance with an embodiment of the present invention. Basket ablation device 40 is fitted at the distal end of shaft 22, and its splines 42 are fitted with ablation electrodes 48, temperature sensors 49 and irrigation apertures 51. Saline solution flowing from irrigation apertures 51 may provide some cooling to nearby tissue.

As seen in FIG. 2, splines 42 are mechanically attached to an extender 44, wherein vibration generator 50 is also fitted to extender 44. Such an arrangement may vibrate splines 42 whenever processor 41 (seen in FIG. 1) is activating vibration generator 50. In certain clinical cases only a limited flow of saline, or none, is allowed. In such cases vibration generator 50 may supplement a larger portion or all of the required cooling capacity. The vibration may induce, for example, mixing of warmer blood with a cooler blood.

In some embodiments, processor 41 may be configured to receive temperature readings from temperature sensors 49, and to adjust the ablation pulses and/or vibration accordingly. Additionally, or alternatively, processor 41 may control vibration generator 50 according to the temperature readings so as to synchronize the vibration with the RF pulses. In some embodiments, processor 41 commands the vibration generator to adjust its vibration-amplitude and/or vibration-frequency responsively to the temperature sensors 49 readings the processor receives, so as to control the temperature. In another embodiment, processor 41 activates or deactivates vibration generator 50 responsively to the temperature sensors 49 readings as to control the temperature.

A vibration coordinated with the RF ablation may further improve cooling. Electrodes 48 motion may allow the endocardial surface of the lesions, otherwise in continuous contact with electrodes 48 and/or blood, to come in contact with cool blood and/or saline, but still remain hot at depth. Vibration of splines 42 and irrigation from apertures 51 may work in synergy to cool tissue otherwise inaccessible to cooling. The disclosed technique may thus have a distinct advantage over non-vibration cooling schemes with regard to the temperature profile it may induce in the tissue, by preventing side effects, such as excessive damage to the surface of the tissue at certain locations, and preventing blood coagulation.

The example configuration shown in FIG. 2 is chosen purely for the sake of conceptual clarity. The disclosed techniques may similarly be applied using any other system components and settings. For example, in an embodiment, system 20 may comprise other sorts of ablation devices, such as a balloon catheter, a circular or lasso-shaped multi-electrode catheter, or a multi-arm multi-electrode catheter.

In an embodiment, vibration generator 50 is implemented by incorporating an electroactive polymer, for example sequestered within the extender rod. The generator of vibration may be an ultrasound generator, or any other device known to the art to create vibration. The power source of the vibration generator can be located at console 24, or at the catheter handle, and wired to the generator through the shaft.

In an embodiment, the vibration generator comprises an oscillating solenoid. Console 24 may supply the alternating electric current to drive the solenoid, through wires running in the catheter shaft. Alternatively, in an embodiment, an external power source could wirelessly drive the vibration generator. For example, when the ablation is performed under MRI imaging, the alternating magnetic fields of the MRI system may induce the alternating current that drives the solenoid to oscillate.

In yet other embodiments, vibrator generator 50 is fitted in the handle of the catheter, instead of at the distal end. In these embodiments, the vibration generator vibrates the proximal end of the catheter, and the vibrations propagate via the entire length of the catheter until ultimately vibrating splines 42.

In some embodiments, the vibration generator vibrates sheath 23, to impart vibration on the catheter distal end, instead of for example, vibrating the ablation device itself. Any other method known to the art to create vibration may be used, which induces oscillations about whatever given axis, for example about the long axis of the shaft, in one of the following forms or any of their combinations: rotation, twist, bending, expansion, contraction, and parallel motion.

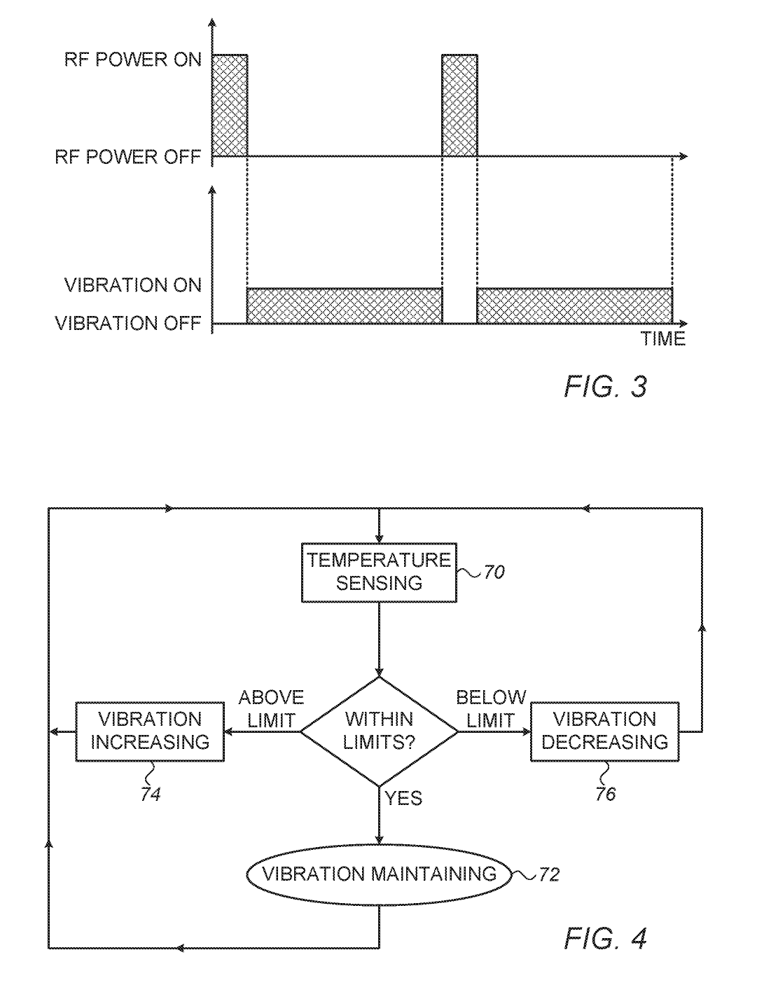

FIG. 3 is a schematic graph illustrating a sequence of synchronized RF energy pulses and vibration pulses (of the ablation device), in accordance with an embodiment of the present invention. As seen, the selected timing scheme for applying the vibration is between the RF ablation pulses. Vibration generator 50 is thus configured to vibrate the ablation device alternately to the RF power. This timing scheme is depicted purely by way of example. In alternative embodiments, processor 41 may apply any other mutual timing or synchronization between the vibrations and the RF ablation pulses.

Synchronizing the vibration to the RF power may cause blood to come into contact briefly with the hot tissue which allows for more immediate evacuation of heat from its surface. Another reason to synchronize that way the vibration with the RF power duty-cycle is that the RF ablation should be applied with a robust mechanical contact between the electrodes to the tissue, namely when the electrodes are statically pressing firmly against the tissue.

The example functionality shown in FIG. 3 is only one of several possible. The disclosed techniques may similarly apply the vibration responsively to reading of temperature, of contact force and flow of irrigation. In yet other embodiments, the RF pulses and the vibrations may be non-synchronized.

FIG. 4 is a flow chart that schematically illustrates a method for controlling tissue temperature during ablation, in accordance with an embodiment of the present invention. As seen, the vibration is controlled in a closed loop. Processor 41 reads the temperature from temperature sensors 49 located in vicinity of ablation electrodes 48, at a temperature sensing step 70. If the read temperatures are within predefined limits then processor 41 does not command any action, and the vibration is maintained as is, as seen at a vibration maintaining step 72.

If one or more of the temperature readings exceed the predefined limits, then processor 41 commands vibration generator 50 to increase vibration, as seen at a vibration increasing step 74. Increasing the vibration may involve, for example, increasing the vibration amplitude, frequency, duration and/or duty-cycle.

If, on the other hand, the read temperatures fall below the predefined limits, processor 41 commands vibration generator 50 to decrease the vibration, as seen at a vibration decreasing step 76. As above, decreasing the vibration may involve decreasing the vibration amplitude, frequency, duration and/or duty-cycle, for example. The method loops back to steps 70 and 72 and proceeds, until the ablation process is completed.

The example flow chart shown in FIG. 4 is chosen purely for the sake of conceptual clarity. In alternative embodiments, the disclosed techniques may use any other suitable control schemes, comprising for example such based on contact force measurements, coordination with a level of flow of irrigation and timing of applicating the RF energy.

Although the embodiments described herein mainly address ablation applications, the methods and systems described herein can also be used in other medical applications.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
a shaft for insertion into a body of a patient;
multiple electrodes, which are fitted at a distal end of the shaft and are configured to deliver Radio Frequency (RF) energy for ablation at multiple respective locations in tissue; and
a vibration generator configured to vibrate the multiple electrodes for providing cooling to the tissue in a vicinity of the locations.

2. The medical instrument according to claim 1, and comprising:
one or more temperature sensors fitted at the distal end of the shaft and configured to measure one or more respective temperatures; and
a processor configured to read the measured temperatures from the temperature sensors and to command the vibration generator to vibrate the multiple electrodes responsively to one or more of the read temperatures.

3. The medical instrument according to claim 2, wherein the processor is configured to adjust at least one of an amplitude and a frequency with which the vibration generator vibrates the multiple electrodes, responsively to one or more of the read temperatures.

4. The medical instrument according to claim 3, wherein the processor is configured to activate or deactivate the vibration generator responsively to one or more of the read temperatures.

5. The medical instrument according to claim 1, wherein the multiple electrodes are configured to deliver the RF energy in a sequence of pulses, and comprising a processor configured to command the vibration generator to vibrate the multiple electrodes in synchronization with the pulses.

6. The medical instrument according to claim 1, and comprising a cooling irrigation device fitted at the distal end of the shaft, wherein the cooling irrigation device is configured to deliver saline solution.

7. The medical instrument according to claim 6, and comprising a processor configured to command the vibration generator to vibrate the multiple electrodes in coordination with delivery of the saline solution from the irrigation device.

8. The medical instrument according to claim 1, and comprising a basket ablation device, a multi-arm ablation device, or a balloon ablation device, which is fitted at the distal end of the shaft and comprises the multiple electrodes.

9. The medical instrument according to claim 1, wherein the vibration generator comprises an electroactive polymer.

10. The medical instrument according to claim 1, wherein the vibration generator comprises an oscillating solenoid.

11. The medical instrument according to claim 10, wherein the oscillating solenoid is configured to be driven by an external alternating magnetic field.

12. The medical instrument according to claim 1, wherein the vibration generator is fitted at the distal end of the shaft.

13. The medical instrument according to claim 1, wherein the vibration generator is fitted at a proximal end of the shaft.

14. The medical instrument according to claim 1, wherein the vibration generator is configured to vibrate the multiple electrodes by vibrating a sheath in which the shaft is inserted.

15. A method, comprising:
inserting a shaft of a medical instrument into a body of a patient;
delivering Radio Frequency (RF) energy from multiple electrodes fitted at a distal end of the shaft for ablating multiple respective locations in a tissue; and
vibrating the multiple electrodes for providing cooling to tissue in a vicinity of the locations.

16. The method according to claim 15, wherein vibrating the multiple electrodes comprises measuring one or more temperatures using one or more respective temperature sensors fitted at the distal end of the shaft, reading the temperatures from the one or more temperature sensors, and vibrating the multiple electrodes responsively to one or more of the read temperatures.

17. The method according to claim 16, wherein vibrating the multiple electrodes comprises adjusting at least one of the amplitude and the frequency with which the multiple electrodes are vibrated, responsively to one or more of the read temperatures.

18. The method according to claim 16, wherein vibrating the multiple electrodes comprises activating or deactivating vibration of the multiple electrodes responsively to one or more of the read temperatures.

19. The method according to claim 15, wherein delivering the RF energy comprises delivering the RF energy in a sequence of pulses, and wherein vibrating the multiple electrodes comprises vibrating the multiple electrodes in synchronization with the pulses.

20. The method according to claim 15, and comprising delivering saline solution during delivery of the RF pulses.

21. The method according to claim 20, wherein vibrating the multiple electrodes comprises vibrating the multiple electrodes in coordination with delivery of the saline solution.

22. The method according to claim 15, wherein vibrating the multiple electrodes comprises applying an external alternating magnetic field.

23. The method according to claim 15, wherein vibrating the multiple electrodes comprises applying vibration at the distal end of the shaft.

24. The method according to claim 15, wherein vibrating the multiple electrodes comprises applying vibration at a proximal end of the shaft.

25. The method according to claim 15, wherein vibrating the multiple electrodes comprises vibrating a sheath in which the shaft is inserted.
